(19) United States
(12) Patent Application Publication
(10) Pub. No.: US 2010/0021397 A1
(43) Pub. Date: Jan. 28, 2010

(54) DROPLET CLEANING FLUID USED FOR CLEANING TEETH WHICH INCLUDES A POLYMER ADDITIVE

(75) Inventors: Paulus Cornelis Duineveld, Beetstarzwaag (NL); William E. Bryant, North Bend, WA (US)

Correspondence Address: PHILIPS INTELLECTUAL PROPERTY & STANDARDS, PO BOX 3001, BRIARCLIFF MANOR, NY, 10510-8001, US

(73) Assignee: KONINKLIJKE PHILIPS ELECTRONICS N.V., EINDHOVEN (NL)

(21) Appl. No.: 11/722,332

(22) PCT Filed: Dec. 20, 2005
(86) PCT No.: PCT/IB05/54339

§ 371 Date: Oct. 13, 2009

Related U.S. Application Data
(60) Provisional application No. 60/638,057, filed on Dec. 21, 2004.

(51) Int. Cl. A61K 8/72; A61Q 11/00

(52) U.S. Cl. 424/49

(57) ABSTRACT

The fluid for use in a liquid droplet-based oral care system for teeth includes a water-soluble, human-consumable polymer additive of selected concentration and molecular weight which maintains the size of the resulting droplets to a desired range of 25-30 microns, at a flow rate within the range of 10-20 milliliters per minute.

This invention relates generally to fluids used in high velocity fluid droplet systems for cleaning teeth, and more particularly concerns such a fluid which includes an additive to maintain a desirable droplet size.

U.S. application Ser. No. 60/537,690 discloses an oral care system for cleaning teeth, including removing dental plaque, by the use of a spray of fine droplets directed at a selected, effective velocity against the teeth. The droplets may be generated in different ways, including forcing a liquid with high pressure through a plurality of nozzles, or by introducing fluid into a fast-flowing stream of air. Another droplet generating embodiment includes a piezoelectric element which produces a high-speed pumping effect on fluid, which accelerates the fluid through a set of nozzles.

Droplet size is important for proper cleansing action, particularly in view of practical operational limitations on flow rate and fluid velocity/pressure. If the droplets are too small, i.e. less than 10 μm, high velocities of up to 200 m/sec are necessary to produce a proper cleaning effect, including plaque removal. Such a high velocity is undesirable, since it dramatically increases the necessary pressure to be produced by the fluid pump, as well as increasing the energy supplied to the liquid. It has heretofore been difficult to produce droplets of desired size for good cleaning without using excessive flow rates or velocities.

Hence, it is desirable to be able to control droplet size while maintaining the droplet flow rate within acceptable limits.

Accordingly, the present invention is a fluid used for cleaning teeth in a droplet-based oral care system, comprising: a fluid which is dispersible into a spray of droplets useful in cleaning teeth in a fluid droplet-based oral care system, wherein the fluid contains a water soluble, human consumable polymer additive of selected molecular weight and in a selected concentration so as to result in a droplet size produced from the resulting additive solution, in a selected range, at least 80% being at least 10 μm in diameter, to produce plaque removal when the droplet spray is directed to the teeth.

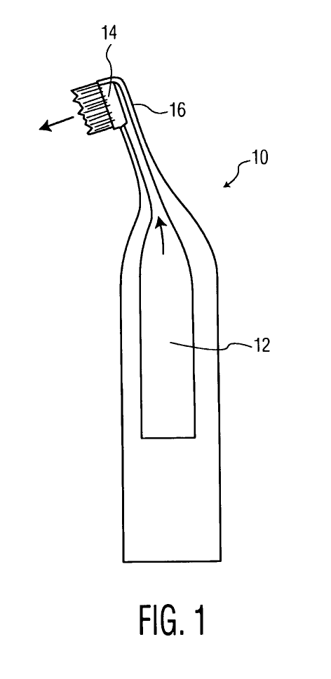

FIG. 1 is a schematic view of an oral care appliance using fluid droplets.

As discussed above, U.S. patent application Ser. No. 60/537,690, filed on Jan. 20, 2004, titled “Droplet Jet System For Cleaning”, which is owned by the assignee of the present invention, the contents of which is hereby incorporated by reference, is directed toward a fluid droplet spray system for cleaning teeth. As disclosed in that application, the fluid droplets may be generated by various means.

It is important for convenient operation that the flow rate of the fluid be limited. Preferably, the fluid flow rate is approximately 20 ml per brushing event, with at a flow rate of 10 ml/min for a two-minute brushing. This is a typical convenient mouth capacity for a user for a brushing event. The flow rate could be somewhat higher, however, perhaps up to 100 ml total, without significant inconvenience to the user. A range could be 5-50 ml per minute.

The desired droplet size (diameter) in such an oral care system is 10-30 μm, or more, in some cases up to 100 μm, for efficient plaque removal. In certain droplet-generating systems, the flow rate is within acceptable ranges, but the droplets are too small, while in other systems, the droplet size is within the desired range, but the flow rate is unacceptably large.

In the present invention, a small concentration of a high molecular weight polymer is added to the fluid, such as water, prior to the generation of the droplets, to produce what is referred to herein as an additive solution. The polymer helps in the production of a desired droplet size. The droplets can then be applied with a destined flow rate to achieve convenient, effective cleaning. The additive solution can be used with any droplet-generating system. The high molecular weight polymers can also be added to oral care fluids other than water, such as for instance, mouthwashes.

The added polymer may be any one of a wide variety of possible polymers. The two general requirements are that the polymer must be water-soluble, and must be consumable, i.e. safe for human consumption. The possible polymers include both “stiff” polymers, such as Xanthan gum or “flexible” polymers, such as polyethylene oxide. Both the molecular weight of the selected polymer and the concentration of the polymer in the fluid of origin are important. The concentration of the high molecular weight polymer should be relatively small so as to limit any increase of shear viscosity of the resulting additive solution. This is discussed in more detail below. The molecular weight of the polymer must be high enough that even at desired small concentrations, the elongational viscosity of the resulting additive solution increases significantly relative to the fluid of origin. The elongational viscosity is the resistance of the fluid to an elongational flow.

For instance, any Newtonian fluid of origin such as water will have an elongational viscosity of three times its shear viscosity. This results in droplets which break up sufficiently easily during formation that the resulting droplets are too small for oral care systems. When a small concentration of a high molecular weight polymer is added to the liquid of origin, the liquid becomes non-Newtonian, resulting in, among other effects, an increase in the elongational viscosity to values much greater than three times the shear viscosity. This increased elongational viscosity refers to the characteristic of a fluid which maintains a droplet configuration of a particular size without separating into smaller droplets. Basically, with a high elongational viscosity, existing droplets “stretch” considerably before separating into smaller droplets. A high elongational viscosity thus provides the desired result of maintaining larger droplet sizes during droplet generation. It has been discovered that the molecular weight of the added polymer should be sufficiently high that the elongational viscosity of the additive solution be approximately six or more times the shear viscosity of the additive solution to produce the desired droplet size.

The effect of the high molecular weight polymer, as indicated above, on the fluid of origin is to decrease the tendency of the resulting additive solution to break up into small droplets, i.e. the size of droplets which would be otherwise produced during droplet generation. This is accomplished by the increase in elongational viscosity of the additive solution accompanied by only a relatively small increase in shear viscosity, due to the small concentration of the high molecular weight polymer additive.

In addition, the high molecular weight polymer prevents the formation of satellite droplets during droplet generation, and further, decreases the drag of the fluid during turbulent flow, which occurs in one or more of the droplet generation systems described in the 60/537,690 application, particularly those embodiments using nozzles.

Xanthan gum, which is one of the example polymers, has a molecular weight of approximately 7.6×10⁶ g/mol; polyethylene oxide, which is another example, can be made in a wide variety of molecular weights. The minimum molecular weight for this application is on the order of 0.1×10⁶ g/mol. Both of these polymers will produce the desired result. The molecular weight of the additive polymer, as explained above, must be sufficiently high as to substantially increase the elongational viscosity of the resulting additive solution, with the concentration of the additive polymer being quite small such that any increase in shear viscosity of the additive solution is also small. The droplets produced are substantially all (at least 80%) approximately at least 10 micrometers in diameter. Typically, the percentage is much closer to 100%.

A particular term “intrinsic” viscosity [μ] of a solution is defined herein as:

$$[ μ ] = {lim}_{c - \to 0} \frac{μ - {μ}_{s}}{{μ}_{s} c}$$

where c is the concentration in mass per unit volume of original fluid, μ is the viscosity of the additive solution and μₛ is the viscosity of the original fluid. At a concentration c* which is larger than 1/[μ]), the shear viscosity of the additive solution will be significantly influenced, which can be undesirable.

For the additive polymer to be effective, the concentration should be between 0.1c*-10c*, but more preferably is in the range 0.25c*-2.5c*.

For xantham gum and water, c*=0.0045% weight has been found to be effective.

A cleaning system involving the above-described fluid includes a power toothbrush, such as shown in FIG. 1, generally at 10. The toothbrush includes a droplet-generating system, shown generally at 12. The fluid containing the polymer additive in operation is forced out through nozzles 14 in the brushhead 16. The fluid coming out of the brushhead breaks up into a fine spray, at a desired flow rate and velocity, as well as with a desired droplet size controlled to an extent by the high molecular weight polymer additive. The spray is directed to the teeth, resulting in cleaning of the teeth, in particular plaque removal.

Although a preferred embodiment of the invention has been disclosed for purposes of illustration, it should be understood that various changes, modifications and substitutions may be incorporated in the embodiment without departing from the spirit of the invention which is defined by the claims which follow.

1. A fluid used for cleaning teeth in a droplet-based oral care system, comprising:
a fluid which is dispersible into a spray of droplets useful in cleaning teeth in a fluid droplet-based oral care system, wherein the fluid contains a water soluble, human consumable polymer additive of selected molecular weight and in a selected concentration so as to result in a droplet size produced from the resulting additive solution, in a selected range, at least 80% of the droplets are at least 10 μm in diameter, to produce plaque removal when the droplet spray is directed to the teeth.

2. The fluid of claim 1, wherein the flow rate of the fluid is 5-50 millimeters per minute.

3. The fluid of claim 1, wherein the molecular weight of the polymer additive is sufficiently high and where the concentration is sufficiently small that the elongational viscosity of the additive solution is at least six times the shear viscosity thereof.

4. The fluid of claim 1, wherein the molecular weight of the polymer additive is at least 0.1×10⁶ grams per molecule.

5. The fluid of claim 1, wherein the concentration of the polymer is between 0.1c*-10c*, where c* is 1/[μ] and
$$[ μ ] = {lim}_{c - \to 0} \frac{μ - {μ}_{s}}{{μ}_{s} c}$$
Where c is the concentration in mass per volume of the original fluid, μ is the viscosity of the resulting additive solution, and μₛ is the viscosity of the original fluid.

6. The system of claim 1, wherein the droplet size is in the range of 10-100 μm.

7. A system for cleaning teeth using a stream of fluid droplets, comprising:
a source of fluid, the fluid including a water-soluble human-consumable polymer additive of selected concentration and molecular weight;
a fluid droplet generator for creating a stream of droplets from the fluid, wherein the droplets have a size in a selected range, at least 80% of the droplets being at least 10 micrometers in diameter; and
a member for directing the stream of droplets onto a selected area of the teeth.

8. The system of claim 7, wherein the flow rate of the fluid is within the range of 5-50 milliliters per minute.

9. The system of claim 7, wherein the droplets are within the range of 10-100 micrometers in diameter.
